(19) United States
(12) Patent Application Publication
(10) Pub. No.: US 2012/0109243 A1
(43) Pub. Date: May 3, 2012

(54) HEART FAILURE MONITORING AND NOTIFICATION

(75) Inventors: Douglas A. Hettrick, Andover, MN (US); Shantanu Sarkar, Roseville, MN (US); Kevin T. Ousdigian, Shoreview, MN (US)

(73) Assignee: Medtronic, Inc., Minneapolis, MN (US)

(21) Appl. No.: 12/914,836

(22) Filed: Oct. 28, 2010

(51) Int. Cl. A61N 1/365; A61B 5/0402

(52) U.S. Cl. 607/17; 600/509

(57) ABSTRACT

Techniques for generating a heart failure risk score with detected patient metrics are described. An implantable medical device (IMD) may collect and store patient data regarding therapy use statistics, thoracic impedance, heart rate, patient activity, and other patient metrics. Based on the number of patient metrics exceeding their respective metric thresholds, the IMD may automatically generate a risk score that indicates the likelihood that the patient will suffer from heart failure. The risk score may identify a patient as requiring immediate medical attention to reduce the risk of heart failure. The IMD may push an alert of the heart failure risk score to a clinician, and the clinician may review the patient metric data on an external device. In some examples, a clinician may prioritize patient treatment with a presented list ranking patients with the most severe heart failure risk scores.

### TECHNICAL FIELD

The invention relates to medical devices, and, more particularly, to medical devices that monitor cardiac health.

### BACKGROUND

Heart failure is a condition affecting thousands of people worldwide. Essentially, congestive heart failure occurs when the heart is unable to pump blood at an adequate rate in response to the filling pressure. This condition may result in congestion in the tissue, peripheral edema, pulmonary edema, and even shortness of breath. When heart failure is severe, it can even lead to patient death.

Although heart failure treatments may include electrical stimulation therapy and drug therapy, drug therapy has been the more effective treatment for most patients. For example, patients suffering from or at risk for heart failure may be treated with diuretic agents and/or angiotensin converting enzyme inhibitors. In addition, patients may be treated with nitroglycerin to reduce the symptoms of heart failure. Even though treatments are available, patients with other cardiac conditions may be at greater risk of severe complications with the conditions of heart failure.

### SUMMARY

Generally, this disclosure describes techniques for generating a heart failure risk score and presenting the risk score to a clinician. An implantable medical device (IMD), e.g., a pacemaker, cardioverter and/or defibrillator, or a monitor that does not provide therapy, may collect and store patient data regarding therapy use statistics (e.g., pacing or shocks), thoracic impedance, heart rate, heart rate variability, patient activity, atrial arrhythmias, and other patient metrics. These metrics may be sensed or collected from electrodes, activity sensors, or any other sensing devices. Each patient metric may be compared to respective metric thresholds for each metric during an evaluation window. Based on the number of patient metrics exceeding their respective metric thresholds, the IMD may automatically generate a risk score that indicates the likelihood that the patient will suffer a heart failure event, e.g., a heart failure decompensation event requiring hospitalization. In other words, when a predetermined number of metrics exceed thresholds, the risk score indicates a high likelihood that the patient will suffer a heart failure event. The risk score may help a clinician or other healthcare professional to identify when a patient requires medical attention to reduce the risk of a heart failure event and other complications.

The risk score and other patient metric information may be delivered to healthcare professionals in different manners. For example, the IMD may push an alert or the heart failure risk score remotely to a clinician when immediate medical attention is needed. In other examples, the clinician may review a heart failure report that includes a trend summary of patient metrics exceeding their threshold and/or all patient metrics with those metrics exceeding thresholds highlighted for ease of diagnosis. In some examples, a remote computing device may receive heart failure risk scores from the IMDs of multiple patients. The heart failure risk scores may be used to rank each patient according to their risk score. Therefore, a list of the ranked patients may be presented to a clinician so that the clinician may prioritize those patients requiring treatment first.

In one example, the disclosure describes a method that includes storing a plurality of automatically detected patient metrics within an implantable medical device and comparing each of the plurality of automatically detected patient metrics to one of a plurality of metric specific thresholds. The method also includes automatically generating a heart failure risk score based on the comparison, wherein the heart failure risk score indicates an increased risk of heart failure when a predetermined number of the plurality of automatically detected patient metrics each exceed the respective one of the plurality of metric specific thresholds.

In another example, the disclosure describes a system that includes a memory of an implantable medical device that stores a plurality of automatically detected patient metrics and a metric detection module configured to compare each of the plurality of automatically detected patient metrics to one of a plurality of metric specific thresholds and automatically generate a heart failure risk score based on the comparison. The heart failure risk score indicates an increased risk of heart failure when a predetermined number of the plurality of automatically detected patient metrics each exceed the respective one of the plurality of metric specific thresholds.

In another example, the disclosure describes a system that includes means for storing a plurality of automatically detected patient metrics within an implantable medical device and means for comparing each of the plurality of automatically detected patient metrics to one of a plurality of metric specific thresholds. The system also includes means for automatically generating a heart failure risk score based on the comparison, wherein the heart failure risk score indicates an increased risk of heart failure when a predetermined number of the plurality of automatically detected patient metrics each exceed the respective one of the plurality of metric specific thresholds.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual drawing illustrating an example system configured to store patient metrics used to generate a heart failure risk score that includes an implantable medical device (IMD) coupled to implantable medical leads.

FIG. 2A is a conceptual drawing illustrating the example IMD and leads of FIG. 1 in conjunction with a heart.

FIG. 2B is a conceptual drawing illustrating the example IMD of FIG. 1 coupled to a different configuration of implantable medical leads in conjunction with a heart.

FIG. 3 is a functional block diagram illustrating an example configuration of the IMD of FIG. 1.

FIG. 4 is a functional block diagram illustrating an example configuration of an external programmer that facilitates user communication with the IMD.

FIG. 5 is a block diagram illustrating an example system that includes an external device, such as a server, and one or more computing devices that are coupled to the IMD and programmer shown in FIG. 1 via a network.

FIG. 6 illustrates an example user interface that includes a trend summary of patient metrics indicating a risk of heart failure.

FIG. 7 illustrates an example user interface that includes data from a plurality of patient metrics used to generate the heart failure risk score.

FIG. 8 illustrates an example user interface that includes a list of patients ranked by severity of their heart failure risk score.

FIG. 9 is a flow diagram of an example method for generating heart failure risk scores from patient metrics.

FIG. 10 is a flow diagram of an example method for presenting heart failure risk scores and patient metric data to a user.

FIG. 11 is a flow diagram of an example method for presenting a user with a ranked list of patients based on the heart failure risk score of each patient.

### DETAILED DESCRIPTION

This disclosure generally describes techniques for generating a heart failure risk score, which may be presented to a clinician for the purpose of reviewing the patient condition and/or treating the patient. Congestive heart failure may occur gradually over time due to heart disease, patient inactivity, cardiac arrhythmias, hypertension, and other conditions. Often, however, a relatively rapid worsening of the patient's condition, e.g., a decompensation, precipitates hospitalization and, in some cases, death. Although it may not be possible for health care professionals to continually monitor patients for potential risk of a heart failure event, e.g., decompensation, certain conditions may be automatically monitored and used to create a heart failure risk score that a clinician may review periodically or transmitted to a clinician when the heart failure risk score indicates that the patient is at an increased risk for a heart failure event.

An implantable medical device (IMD), e.g., a pacemaker, cardioverter and/or defibrillator, may collect and store patient data regarding patient metrics, such as therapy use statistics (e.g., pacing or shock delivery), thoracic impedance, heart rate, heart rate variability, and patient activity. Other example patient metrics include weight, blood pressure, respiration rate, sleep apnea burden derived from respiration rate, temperature, ischemia burden, cardiac events, and sensed cardiac event intervals. Example cardiac events may include atrial fibrillation, ventricular rate during atrial fibrillation, or ventricular tachyarrhythmias. The concentration or levels of various substances, such as troponin and/or brain natriuretic peptide (BNP) levels, within the patient may also be patient metrics.

These metrics may be sensed and/or collected from electrodes, activity sensors, or any other sensing devices to detect the patient's risk of heart failure. For each patient metric, a metric threshold may be used to detect when the metric has reached a serious state. In this manner, each patient metric may be compared to respective thresholds during an evaluation window. The IMD may then automatically generate a heart failure risk score that indicates the likelihood of a heart failure event based on the number of patient metrics exceeding their respective metric thresholds. As used herein, the term “score” may refer to a numerical score, or other types of scores, such as levels, e.g., high, medium, or low risk of heart failure.

When a predetermined number of patient metrics each exceed their threshold, the risk score indicates a high likelihood that the patient will suffer from heart failure. This predetermined number of patient metrics may be set as a percentage or fraction, e.g., any number of metrics out of the total number of monitored metrics. In one example, a predetermined number of only two exceeded metrics out of eight total metrics may indicate treatment is required. In another example, each of the metrics may be weighted differently such that the weighted sum of threshold-exceeding metrics may be compared to a threshold to determine whether a heart failure event is probable. In this manner, those metrics having a greater impact on heart failure risk may have a greater impact on the risk score. The risk score may help a clinician or other healthcare professional to identify when a patient requires medical attention to reduce the risk of a heart failure event or other complications.

The risk score and other patient metric information may be delivered to healthcare professionals through different methods and different channels. For example, the IMD may push or send an alert of the heart failure risk score remotely (e.g., wired or wireless transmission methods) to a clinician when predetermined number for the risk score is reached. In other examples, the clinician may review a heart failure report that includes a trend summary of patient metrics exceeding the respective thresholds and/or all patient metric data with those metrics exceeding thresholds highlighted for ease of diagnosis. In some examples, a remote computing device, e.g., a clinic server or workstation, may receive heart failure risk scores from the IMDs of multiple patients. The heart failure risk scores may be used to rank each patient according to their risk score. Therefore, a list of the ranked patients may be presented to a clinician so that the clinician may prioritize those patients at a higher risk for heart failure.

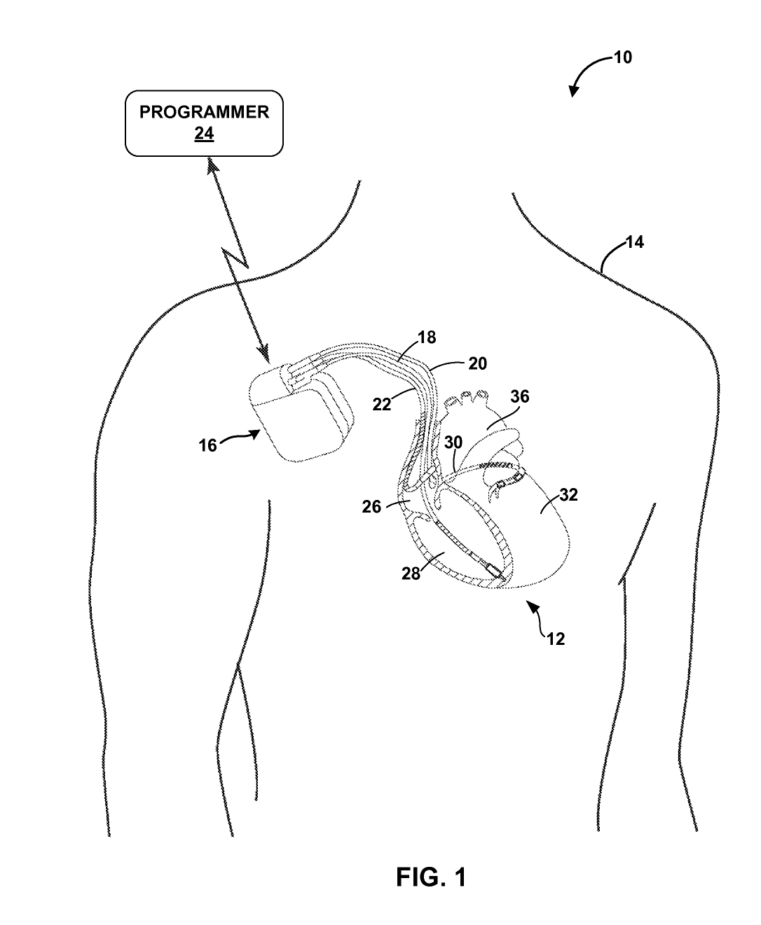

FIG. 1 is a conceptual drawing illustrating an example system 10 configured to detect patient metrics used to automatically generate heart failure risk scores for patient 14. In the example of FIG. 1, system 10 includes IMD 16, which is coupled to leads 18, 20, and 22, and programmer 24. IMD 16 may be, for example, an implantable pacemaker, cardioverter, and/or defibrillator that provides electrical signals to heart 12 via electrodes coupled to one or more of leads 18, 20, and 22. Patient 14 is ordinarily, but not necessarily a human patient.

Although an implantable medical device and delivery of electrical stimulation to heart 12 are described herein as examples, the techniques for detecting patient metrics and generating heart failure risk scores of this disclosure may be applicable to other medical devices and/or other therapies. In general, the techniques described in this disclosure may be implemented by any medical device, e.g., implantable or external, that senses a signal indicative of cardiac activity, patient 14 activity, and/or fluid volume within patient 14. As one alternative example, the techniques described herein may be implemented in an external cardiac monitor that generates electrograms of heart 12 and detects thoracic fluid volumes of patient 14.

In the example of FIG. 1, leads 18, 20, 22 extend into the heart 12 of patient 14 to sense electrical activity of heart 12 and/or deliver electrical stimulation to heart 12. Leads 18, 20, and 22 may also be used to detect a thoracic impedance indicative of fluid volume in patient 14, respirations rates, sleep apnea, or other patient metrics. Respiration rates and sleep apnea may also be detectable via an electrogram. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

In some examples, system 10 may additionally or alternatively include one or more leads or lead segments (not shown in FIG. 1) that deploy one or more electrodes within the vena cava, or other veins. Furthermore, in some examples, system 10 may additionally or alternatively include temporary or permanent epicardial or subcutaneous leads with electrodes implanted outside of heart 12, instead of or in addition to transveous, intracardiac leads 18, 20 and 22. Such leads may be used for one or more of cardiac sensing, pacing, or cardioversion/defibrillation. For example, these electrodes may allow alternative electrical sensing configurations that provide improved or supplemental sensing in some patients. In other examples, these other leads may be used to detect intrathoracic impedance as a patient metric for identifying a heart failure risk.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may detect arrhythmia of heart 12, such as tachycardia or fibrillation of the atria 26 and 36 and/or ventricles 28 and 32, and may also provide defibrillation therapy and/or cardioversion therapy via electrodes located on at least one of the leads 18, 20, 22. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In addition, IMD 16 may monitor the electrical signals of heart 12 for patient metrics used in generating the heart failure risk score. IMD 16 may utilize two of any electrodes carried on leads 18, 20, 22 to generate electrograms of cardiac activity. In some examples, IMD 16 may also use a housing electrode of IMD 16 (not shown) to generate electrograms and monitor cardiac activity. Although these electrograms may be used to monitor heart 12 for potential arrhythmias and other disorders for therapy, the electrograms may also be used to monitor the condition of heart 12. For example, IMD 16 may monitor heart rate (night time and day time), heart rate variability, ventricular or atrial intrinsic pacing rates, indicators of blood flow, or other indicators of the ability of heart 12 to pump blood or the progression of heart failure.

In some examples, IMD 16 may also use any two electrodes of leads 18, 20, and 22 or the housing electrode to sense the intrathoracic impedance of patient 14. As the tissues within the thoracic cavity of patient 14 increase in fluid content, the impedance between two electrodes may also change. For example, the impedance between an RV coil electrode and the housing electrode may be used to monitor changing intrathoracic impedance. An example system for measuring thoracic impedance is described in U.S. Pat. No. 6,104,949 to Pitts Crick et al., entitled, “MEDICAL DEVICE,” which issued on Aug. 15, 2000 and is incorporated herein by reference in its entirety. IMD 16 may use this impedance to create a fluid index. As the fluid index increases, more fluid is being retained within patient 14 and heart 12 may be stressed to keep up with moving the greater amount of fluid. Therefore, this fluid index may be a patient metric used to generate the heart failure risk score. By monitoring the fluid index in addition to other patient metrics, IMD 16 may be able to reduce the number of false positive heart failure identifications from monitoring only one or two patient conditions.

IMD 16 may also communicate with external programmer 24. In some examples, programmer 24 comprises a handheld computing device, computer workstation, or networked computing device. Programmer 24 may include a user interface that receives input from a user. In other examples, the user may also interact with programmer 24 remotely via a networked computing device. The user may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of IMD 16. Although the user is a physician, technician, surgeon, electrophysiologist, or other healthcare professional, the user may be patient 14 in some examples.

For example, the user may use programmer 24 to retrieve information from IMD 16 regarding patient metric data and/or the heart failure risk score. Although programmer 24 may retrieve this information, IMD 16 may push or transmit the heart failure risk score if the heart failure risk score reaches a predetermined number of patient metrics exceeding their representative thresholds. Although IMD 16 may generate the heart failure risk score, IMD 16 may transmit the patient metric data and programmer 24 may generate the heart failure risk score in other examples. Programmer 24 may present an alert to the user with the heart failure risk score and/or other patient metric data. This patient metric data may include intracardiac or intravascular pressure, activity, posture, respiration, or thoracic impedance. As another example, the user may use programmer 24 to retrieve information from IMD 16 regarding the performance or integrity of IMD 16 or other components of system 10, such as leads 18, 20 and 22, or a power source of IMD 16. In some examples, any of this information may be presented to the user as an alert (e.g., a notification or instruction). Further, alerts may be pushed from IMD 16 to facilitate alert delivery whenever programmer 24 is detectable by IMD 16. IMD 16 may wirelessly transmit alerts to facilitate immediate notification of the heart failure condition.

Programmer 24 may also allow the user to define how IMD 16 senses, detects, and manages each of the patient metrics. For example, the user may define the frequency of sampling or the evaluation window used to monitor the patient metrics. In addition, the user may use programmer 24 to set each metric threshold used to monitor the status of each patient metric. The metric thresholds may be used to determine when each of the patient metrics has reached a magnitude indicative of being at risk for heart failure. When a patient metric exceeds its respective metric threshold, the metric may be counted in the predetermined number used to create the heart failure risk score. For example, if two of the eight patient metrics exceed their thresholds, the heart failure risk score may be set to 2/8. This heart failure risk score may indicate that patient 14 is at an increased risk of heart failure if the predetermined number of the risk score is set to two. This predetermined number may be set to different values for patients of differing age, weight, cardiac condition, or any number of other risk factors. In other examples, the predetermined number may be set to a different number or a risk score percentage (fraction) may be used instead such that the predetermined number represents a preset fraction of unweighted or weighted metrics exceeding threshold with respect to the total number of monitored metrics. Programmer 24 may be used to set this predetermined number or any other factors used to generate and interpret the heart failure risk score.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

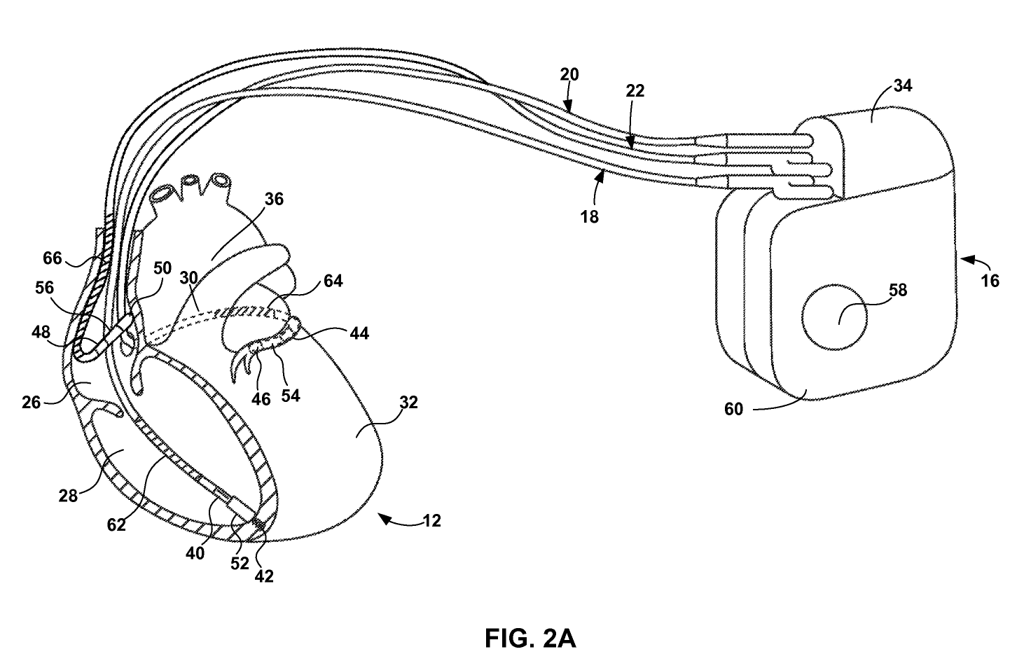

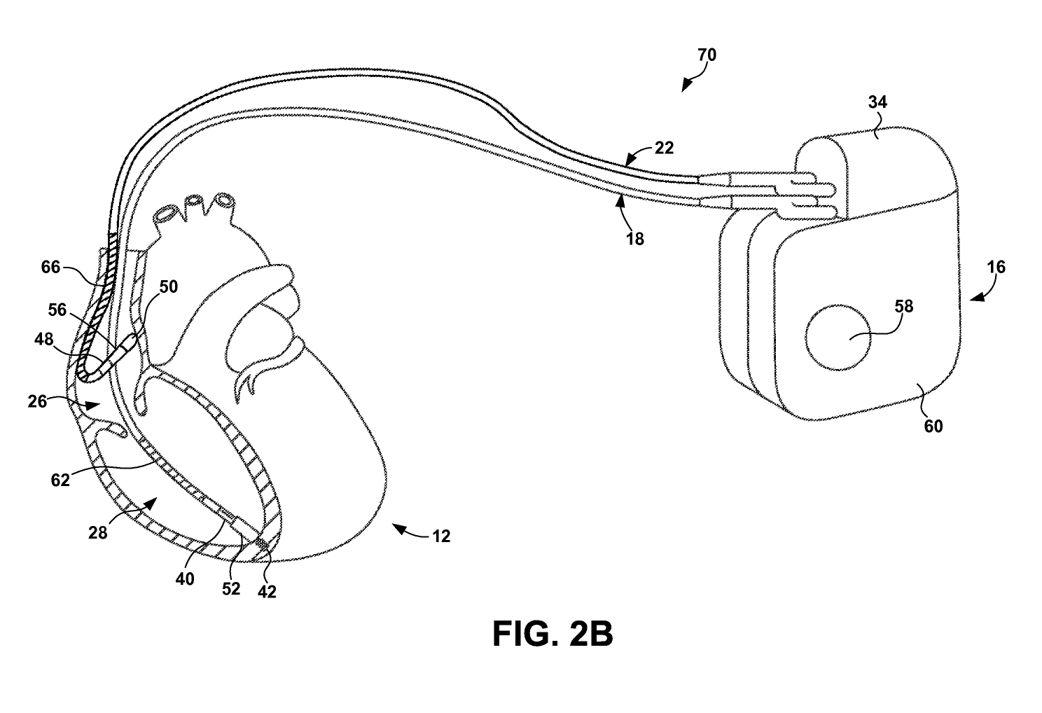

FIG. 2A is a conceptual drawing illustrating IMD 16 and leads 18, 20, and 22 of system 10 in greater detail. As shown in FIG. 2A, IMD 16 is coupled to leads 18, 20, and 22. Leads 18, 20, 22 may be electrically coupled to a signal generator, e.g., stimulation generator, and a sensing module of IMD 16 via connector block 34. In some examples, proximal ends of leads 18, 20, 22 may include electrical contacts that electrically couple to respective electrical contacts within connector block 34 of IMD 16. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. In the illustrated example, there are no electrodes located in left atrium 36. However, other examples may include electrodes in left atrium 36.

Electrodes 40, 44 and 48 may take the form of ring electrodes, and electrodes 42, 46 and 50 may take the form of extendable helix tip electrodes mounted retractably within insulative electrode heads 52, 54 and 56, respectively. In other examples, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

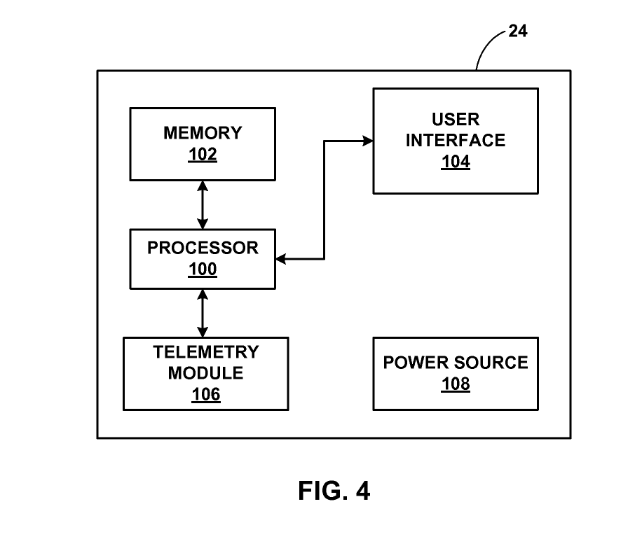

In some examples, as illustrated in FIG. 2A, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 4, housing 60 may enclose a signal generator that generates therapeutic stimulation, such as cardiac pacing pulses and defibrillation shocks, as well as a sensing module for monitoring the rhythm of heart 12.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration or electrode vector.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. The combination of electrodes used for delivery of stimulation or sensing, their associated conductors and connectors, and any tissue or fluid between the electrodes, may define an electrical path.

The configuration of system 10 illustrated in FIGS. 1 and 2A is merely one example. In other examples, a system may include epicardial leads and/or subcutaneous electrodes instead of or in addition to the transveous leads 18, 20, 22 illustrated in FIG. 1. Further, IMD 16 need not be implanted within patient 14. In examples in which IMD 16 is not implanted in patient 14, IMD 16 may sense electrical signals and/or deliver defibrillation pulses and other therapies to heart 12 via percutaneous leads that extend through the skin of patient 14 to a variety of positions within or outside of heart 12. Further, external electrodes or other sensors may be used by IMD 16 to deliver therapy to patient 14 and/or sense and detect patient metrics used to generate a heart failure risk score.

In addition, in other examples, a system may include any suitable number of leads coupled to IMD 16, and each of the leads may extend to any location within or proximate to heart 12. For example, other examples of systems may include three transvenous leads located as illustrated in FIGS. 1 and 2, and an additional lead located within or proximate to left atrium 36. As another example, other examples of systems may include a single lead that extends from IMD 16 into right atrium 26 or right ventricle 28, or two leads that extend into a respective one of the right ventricle 26 and right atrium 26. An example of a two lead type of system is shown in FIG. 2B. Any electrodes located on these additional leads may be used in sensing and/or stimulation configurations.

Any of electrodes 40, 42, 44, 46, 48, 50, 62, 64, 66, and 58 may be utilized by IMD 16 to sense or detect patient metrics used to generate the heart failure risk score for patient 14. Typically, IMD 16 may detect and collect patient metrics from those electrode vectors used to treat patient 14. For example, IMD 16 may derive an atrial fibrillation duration, heart rate, and heart rate variability metrics from electrograms generated to deliver pacing therapy. However, IMD 16 may utilize other electrodes to detect these types of metrics from patient 14 when other electrical signals may be more appropriate for therapy.

In addition to electrograms of cardiac signals, any of electrodes 40, 42, 44, 46, 48, 50, 62, 64, 66, and 58 may be used to sense non-cardiac signals. For example, two or more electrodes may be used to measure an impedance within the thoracic cavity of patient 14. This intrathoracic impedance may be used to generate a fluid index patient metric that indicates the amount of fluid building up within patient 14. Since a greater amount of fluid may indicate increased pumping loads on heart 12, the fluid index may be used as an indicator of heart failure risk. IMD 16 may periodically measure the intrathoracic impedance to identify a trend in the fluid index over days, weeks, months, and even years of patient monitoring.

In general, the two electrodes used to measure the intrathoracic impedance may be located at two different positions within the chest of patient 14. For example, coil electrode 62 and housing electrode 58 may be used as the sensing vector for intrathoracic impedance because electrode 62 is located within RV 28 and housing electrode 58 is located at the IMD 16 implant site generally in the upper chest region. However, other electrodes spanning multiple organs or tissues of patient 14 may also be used, e.g., an additional implanted electrode used only for measuring thoracic impedance.

FIG. 2B is a conceptual diagram illustrating another example system 70, which is similar to system 10 of FIGS. 1 and 2, but includes two leads 18, 22, rather than three leads. Leads 18, 22 are implanted within right ventricle 28 and right atrium 26, respectively. System 70 shown in FIG. 2B may be useful for physiological sensing and/or providing pacing, cardioversion, or other therapies to heart 12. Detection and classification of ischemia according to this disclosure may be performed in two lead systems in the manner described herein with respect to three lead systems. In other examples, a system similar to systems 10 and 70 may only include one lead (e.g., any of leads 18, 20 or 22) to deliver therapy and/or sensor and detect patient metrics related to monitoring risk of heart failure.

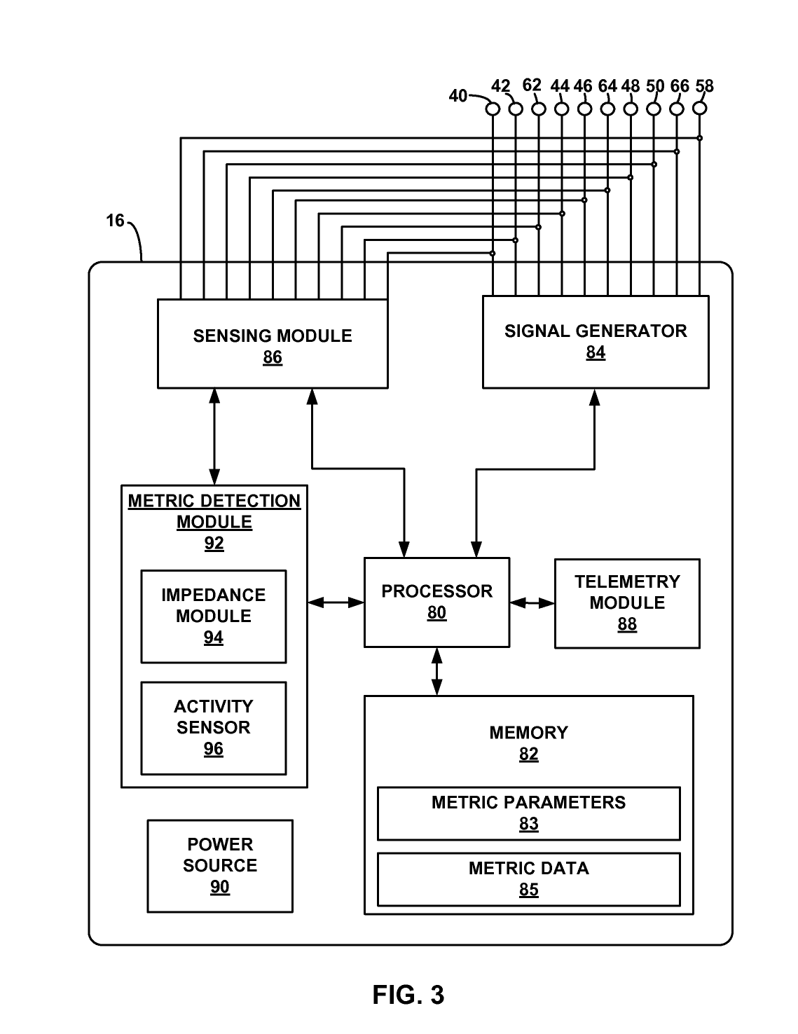

FIG. 3 is a functional block diagram illustrating an example configuration of IMD 16. In the illustrated example, IMD 16 includes a processor 80, memory 82, metric detection module 92, signal generator 84, sensing module 86, telemetry module 88, and power source 90. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or analog logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

Processor 80 controls signal generator 84 to deliver stimulation therapy to heart 12 according to a selected one or more of therapy programs, which may be stored in memory 82. For example, processor 80 may control stimulation generator 84 to deliver electrical pulses with the amplitudes, pulse widths, frequency, or electrode polarities specified by the selected one or more therapy programs.

Signal generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In the illustrated example, signal generator 84 is configured to generate and deliver electrical stimulation therapy to heart 12. For example, signal generator 84 may deliver defibrillation shocks to heart 12 via at least two electrodes 58, 62, 64, 66. Signal generator 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator may deliver one or more of these types of stimulation in the form of other signals, such as sine waves, square waves, or other substantially continuous time signals.

Signal generator 84 may include a switch module and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Electrical sensing module 86 monitors signals from at least one of electrodes 40, 42, 44, 46, 48, 50, 58, 62, 64 or 66 in order to monitor electrical activity of heart 12, impedance, or other electrical phenomenon. Sensing may be done to determine heart rates, heart rate variability, arrhythmias, or other electrical signals. Sensing module 86 may also include a switch module to select which of the available electrodes are used to sense the heart activity, depending upon which electrode combination, or electrode vector, is used in the current sensing configuration. In some examples, processor 80 may select the electrodes that function as sense electrodes, i.e., select the sensing configuration, via the switch module within sensing module 86. Sensing module 86 may include one or more detection channels, each of which may be coupled to a selected electrode configuration for detection of cardiac signals via that electrode configuration. Some detection channels may be configured to detect cardiac events, such as P- or R-waves, and provide indications of the occurrences of such events to processor 80, e.g., as described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Processor 80 may control the functionality of sensing module 86 by providing signals via a data/address bus.

Processor 80 may include a timing and control module, which may be embodied as hardware, firmware, software, or any combination thereof. The timing and control module may comprise a dedicated hardware circuit, such as an ASIC, separate from other processor 80 components, such as a microprocessor, or a software module executed by a component of processor 80, which may be a microprocessor or ASIC. The timing and control module may implement programmable counters. If IMD 16 is configured to generate and deliver pacing pulses to heart 12, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control module within processor 80 may include atrial and ventricular pacing escape intervals, refractory periods during which sensed P-waves and R-waves are ineffective to restart timing of the escape intervals, and the pulse widths of the pacing pulses. As another example, the timing and control module may withhold sensing from one or more channels of sensing module 86 for a time interval during and after delivery of electrical stimulation to heart 12. The durations of these intervals may be determined by processor 80 in response to stored data in memory 82. The timing and control module of processor 80 may also determine the amplitude of the cardiac pacing pulses.

Interval counters implemented by the timing and control module of processor 80 may be reset upon sensing of R-waves and P-waves with detection channels of sensing module 86. In examples in which IMD 16 provides pacing, signal generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. In such examples, processor 80 may reset the interval counters upon the generation of pacing pulses by signal generator 84, and thereby control the basic timing of cardiac pacing functions, including anti-tachyarrhythmia pacing.

The value of the count present in the interval counters when reset by sensed R-waves and P-waves may be used by processor 80 to measure the durations of R-R intervals, P-P intervals, P-R intervals and R-P intervals, which are measurements that may be stored in memory 82. Processor 80 may use the count in the interval counters to detect a tachyarrhythmia event, such as VF or VT. These intervals may also be used to detect the overall heart rate, ventricular contraction rate, and heart rate variability. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other arrhythmia detection methodologies may also be employed by processor 80 in other examples.

In some examples, processor 80 may determine that tachyarrhythmia has occurred by identification of shortened R-R (or P-P) interval lengths. Generally, processor 80 detects tachycardia when the interval length falls below 220 milliseconds (ms) and fibrillation when the interval length falls below 180 ms. These interval lengths are merely examples, and a user may define the interval lengths as desired, which may then be stored within memory 82. This interval length may need to be detected for a certain number of consecutive cycles, for a certain percentage of cycles within a running window, or a running average for a certain number of cardiac cycles, as examples.

In the event that processor 80 detects an atrial or ventricular tachyarrhythmia based on signals from sensing module 86, and an anti-tachyarrhythmia pacing regimen is desired, timing intervals for controlling the generation of anti-tachyarrhythmia pacing therapies by signal generator 84 may be loaded by processor 80 into the timing and control module to control the operation of the escape interval counters therein and to define refractory periods during which detection of R-waves and P-waves is ineffective to restart the escape interval counters for the an anti-tachyarrhythmia pacing. In the event that processor 80 detects an atrial or ventricular tachyarrhythmia based on signals from sensing module 86, and a cardioversion or defibrillation shock is desired, processor 80 may control the amplitude, form and timing of the shock delivered by signal generator 84.

Memory 82 may be configured to store a variety of operational parameters, therapy programs, sensed and detected data, and any other information related to the therapy and treatment of patient 14. In the example of FIG. 3, memory 82 also includes metric parameters 83 and metric data 85. Metric parameters 83 may include all of the parameters and instructions required by processor 80 and metric detection module 92 to sense and detect each of the patient metrics used to generate the heart failure risk score. Metric data 85 may store all of the data generated from the sensing and detecting of each patient metric. In this manner, memory 82 stores a plurality of automatically detected patient metrics as the data required to identify increased risk for heart failure.

Metric parameters 83 may include definitions of each of the patient metrics automatically sensed or measured by metric detection module 92. These definitions may include instructions regarding what electrodes or sensors to use in the detection of each metric, the sample rate, calibration schemes, and any other related information. In one example, the patient metrics for which metric parameters are stored as metric parameters 83 may include a thoracic fluid index, an atrial tachycardia or fibrillation burden, a ventricular contraction rate during atrial fibrillation, a patient activity, a nighttime heart rate, a heart rate variability, a cardiac resynchronization therapy percentage, a bradyarrythmia pacing therapy percentage (in a ventricle and/or atrium) and an electrical shock event. In other examples, other patient metrics may be stored that may be useful in the detection of heart failure risk, e.g., blood pressure, lung volume, lung density, and breathing rate. In such examples, IMD 16 may include or be coupled to sensors known in the art for detecting such metrics. In some examples, the atrial tachycardia or fibrillation burden may be a time of the event, a percent or amount of time over a certain period, a number of episodes, or even a frequency of episodes.

Metric parameters 83 may also store a metric specific threshold for each of the patient metrics automatically detected by metric detection module 92. Metric thresholds may be predetermined and held constant over the entire monitoring of patient 14. However, metric thresholds may be modified by a user during therapy or processor 80 may automatically modify one or more metric thresholds to compensate for certain patient conditions. For example, a heart rate threshold may be changed over the course of monitoring if the normal or baseline heart rate has changed during therapy.

In one example, these metric specific thresholds may include a thoracic fluid index threshold of approximately 60 ohms, an atrial fibrillation burden threshold of approximately 6 consecutive hours, a ventricular contraction rate threshold approximately equal to 90 beats per minute for 24 hours, a patient activity threshold approximately equal to 1 hour per day for seven consecutive days, a nighttime heart rate threshold of approximately 85 beats per minute for seven consecutive days, a heart rate variability threshold of approximately 60 milliseconds for seven consecutive days, a cardiac resynchronization therapy percentage threshold of 90 percent for five of seven consecutive days, and an electrical shock threshold of 1 electrical shock. These thresholds may be different in other examples, and may be configured by a user, e.g., a clinician, for an individual patient.

Any time that an automatically detected patient metric exceeds their respective metric threshold, the patient metric is counted in the heart failure risk score. Therefore, if three of the eight patient metrics exceed their respective metric threshold, then the heart failure risk score would be 3 out of 8 (e.g., threshold exceeding metrics out of the total number of monitored metrics). The higher the risk score, the more likely that patient 14 will suffer a heart failure event. For example, each threshold exceeding metric counted in the predetermined number may contribute to a higher risk of heart failure. In this example, a risk score of 1 out of 8 may indicate a 3% probability of heart failure; a risk score of 2 out of 8 may indicate a 14% probability of heart failure, and a risk score of 3 out of 8 may indicate a 43% probability of heart failure. It is also noted that exceeding a metric threshold does not require that the detected value of the patient metric becomes greater than the magnitude of the threshold. For some patient metrics, exceeding the metric threshold may occur when the value of the patient metric drops below the metric threshold. Therefore, each threshold may be merely a boundary that triggers the metric's inclusion in the heart failure risk score any time that the metric threshold is crossed. In other examples, as described above, the risk score may be calculated as a sum of weighted metrics such that some metrics may impact the risk score greater than other metrics (e.g., a trans-thoracic impedance may be weighted double that of other metrics).

Metric parameters 83 may generally store one metric specific threshold per patient metric, but other examples may include several thresholds to apply depending on other patient conditions, delivered therapies, or even the importance of one patient metric. For example, the thoracic fluid index sensed from intrathoracic impedance may be subject to two separate metric thresholds each counting towards the predetermined number of the heart failure risk index. The first thoracic fluid index threshold may be set to 60 ohms, but the second thoracic fluid index threshold may be set to 100 ohms. If the thoracic fluid index metric exceeds the first thoracic fluid index threshold of 60 ohms, the fluid index metric may be counted in the heart failure risk score. If the fluid index also crosses the second thoracic fluid index threshold of 100 ohms, the fluid index metric may be counted in the heart failure risk score again. In this manner, the heart failure risk score may weight extreme values of some metrics more heavily than other metrics.

Metric parameters 83 may also store instructions for generating the heart failure risk score and the predetermined number for when the risk score is transmitted, or pushed, to a clinician. Although the heart failure risk score may be delivered and presented to users at any time, the heart failure risk score may be pushed to a user when it indicates an increased risk of heart failure. The risk score may become critical when the predetermined number of patient metrics each exceed their respective metric specific threshold. For example, if the predetermined number is set at two, then the heart failure risk score becomes critical when two patient metrics each exceed their threshold. Once the heart failure risk score is critical, processor 80 may push the risk score to a user at a remote location since patient 14 requires medical treatment to avoid heart failure or reduce any damage caused by the condition.

Metric data 85 is a portion of memory 82 that may store some or all of the patient metric data that is sensed and detected by metric detection module 92. Metric data 85 may store the data for each metric on a rolling basis and delete old data as necessary or only for a predetermined period of time, e.g., an evaluation window. Processor 80 may access metric data when necessary to retrieve and transmit patient metric data and/or generate heart failure risk scores. Although metric parameters 83 and/or metric data 85 may consist of separate physical memories, these components may simply be an allocated portion of the greater memory 82.

Metric detection module 92 may automatically sense and detect each of the patient metrics required to generate the heart failure risk score. For example, metric detection module 92 may measure the thoracic impedance, analyze an electrogram of heart 12, monitor the electrical stimulation therapy delivered to patient 14, or sense the patient activity. It is noted that functions attributed to metric detection module herein may be embodied as software, firmware, hardware or any combination thereof. In some examples, metric detection module 92 may at least partially be a software processor executed by processor 80. Metric detection module 92 may sense or detect any of the patient metrics used to generate the heart failure risk score or otherwise indicate that patient 14 may be susceptible to heart failure. Metric detection module 92 may also compare each of the patient metrics to their respective metric specific thresholds defined in metric parameters 83. Metric detection module 92 may automatically detect two or more patient metrics. In some examples, metric detection module 92 may detect eight different patient metrics.

In one example, metric detection module 92 may analyze electrograms received from sensing module 86 to detect an atrial fibrillation or atrial tachycardia, and determine atrial tachycardia or fibrillation burden, e.g., duration, as well as a ventricular contraction rate during atrial fibrillation. Metric detection module 92 may also analyze electrograms in conjunction with a real-time clock to determine a nighttime heart rate or a daytime heart rate or a difference between the day and night heart rate, and also analyze electrograms to determine a heart rate variability, or any other detectable cardiac events from one or more electrograms. As described above, metric detection module 92 may use peak detection, interval detection, or other methods to analyze the electrograms.

In addition, metric detection module 92 may include and/or control impedance module 94 and activity sensor 96. Impedance module 94 may be used to detect the thoracic impedance used to generate the thoracic fluid index. As described herein, impedance module 94 may utilize any of the electrodes of FIG. 1, 2 or 3 to take intrathoracic impedance measurements. In other examples, impedance module 94 may utilize separate electrodes coupled to IMD 16 or in wireless communication with telemetry module 88. Once impedance module 94 measures the intrathoracic impedance of patient 14, metric detection module 92 may generate the thoracic fluid index and compare the index to the thoracic fluid index threshold defined in metric parameters 83.

Activity sensor 96 may include one or more accelerometers or other devices capable f detecting motion and/or position of patient 14. Activity sensor 96 may therefore detect activities of patient 14 or postures engaged by patient 14. Metric detection module 92 may then monitor the patient activity metric based on the magnitude or duration of each activity and compare the determined metric data to the activity threshold defined in metric parameters 83. The activity patient metric may then be used to generate the heart failure risk score.

In addition to detecting events of patient 14, metric detection module 92 may also detect certain therapies delivered by processor 80 and signal generator 84. Metric detection module 92 may monitor signals through signal generator 84 or receive therapy information directly from processor 80 for the detection. Example patient metrics detected by this method may include a cardiac resynchronization therapy percentage and an electrical shock event. The cardiac resynchronization therapy percentage may simply be the amount of time each day, for example, that patient 14 receives some kind of electrical stimulation therapy to heart 12. This therapy may come in the form of pacing pulses, cardioversion, and/or defibrillation, for example. Low therapy percentages may indicate that beneficial therapy is not being delivered and that adjustment of therapy parameters, e.g., an atrioventricular delay or a lower pacing rate, may improve therapy efficacy. In one example, higher therapy percentages may indicate that heart 12 is sufficiently pumping blood through the vasculature with the aid of therapy to prevent fluid buildup. In other examples, higher therapy percentages may indicate that heart 12 is unable to keep up with blood flow requirements. An electrical shock may be a defibrillation event or other high energy shock used to return hear 12 to a normal rhythm. Metric detection module 92 may detect these patient metrics as well and compare them to a cardiac resynchronization therapy percentage and shock event threshold, respectively, defined in metric parameters 83 to determine when each patient metric has become critical. In one example, the electrical shock event may become critical is patient 14 even receives one therapeutic shock.

Metric detection module 92 may include additional sub-modules or sub-routines that detect and monitor other patient metrics used to monitor patient 14 and/or generate the heart failure risk score. In some examples, metric detection module 92, or portions thereof, may be incorporated into processor 80 or sensing module 86. In other examples, raw data used to produce patient metric data may be stored in metric data 85 for later processing or transmission to an external device. An external device may then produce each patient metric from the raw data, e.g., electrogram or intrathoracic impedance. In other examples, metric detection module 92 may additionally receive data from one or more implanted or external devices used to detect each metric such that IMD 16 stores the metric data.

In some examples, the patient metric thresholds may change over time, either modified by a user or automatically changed based on other patient conditions. Telemetry module 88 may receive commands from programmer 24, for example, to modify one or more metric parameters 83 (e.g., metric creation instructions or metric specific thresholds). Alternatively, processor 80 may automatically adjust a metric specific threshold if certain conditions are present in patient 14. For example, the threshold may be adjusted if patient 14 is experiencing certain arrhythmias or normal electrograms change in a manner that requires a change in the threshold

Processor 80 may generate the heart failure risk score based upon the patient metrics sensed, detected, and stored in observation data 85 of memory 82. For example, processor 80 may continually update the heart failure risk score as metric detection module 92 updates each patient metric. In other examples, processor 80 may periodically update the heart failure risk score according to an updating schedule. Processor 80 may compare each of the automatically detected patient metrics their respective metric specific thresholds and automatically generate the heart failure risk score based on the comparison.

Processor 80 may also compare the heart failure risk score to the predetermined number stored in memory 82. The predetermined number may indicate when patient 14 is at an increased risk of heart failure. The predetermined number may be a percentage or a number of patient metrics exceeding the respective metric threshold. At this stage, the risk score may be considered critical. Although a clinician may be presented with the heart failure risk score at any time, processor 80 may push the heart failure risk score to a clinician or other healthcare professional in an alert. This immediacy may be necessary because a critical risk score indicates that heart failure may be imminent in a large number of patients with the same patient metric levels.

In other examples, the heart failure risk score may be generated with a processor of an external computing device, e.g. programmer 24 or external server. However, processor 80 may still collect and store the data for each patient metric or even organize and format the patient metric data before transmitting the patient metrics in metric data 85 to the external device. In addition, processor 80 may transmit the metric thresholds with the patient metric data so that any external device may generate heart failure risk scores specific to patient 14.

As described above, processor 80 may provide an alert to a user, e.g., of programmer 24, regarding the data from any patient metric and/or the heart failure risk score. In one example, processor 80 may provide an alert with the heart failure risk score when programmer 24 or another device communicates with IMD 16. In other examples, processor 80 may push an alert to programmer 24 or another device whenever the heart failure risk score becomes critical via transmission by telemetry module 88. Alternatively, IMD 16 may directly indicate to patient 14 that medical treatment is needed due to a critical heart failure risk score. IMD 16 may include a speaker to emit an audible sound through the skin of patient 14 or a vibration module that vibrates to notify patient 14 of needed medical attention. Processor 80 may choose this action, for example, if the alert cannot be sent because of no available connection.

Telemetry module 88 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

In some examples, processor 80 may transmit atrial and ventricular heart signals, e.g., EGMs, produced by atrial and ventricular sense amplifier circuits within sensing module 86 to programmer 24. Programmer 24 may interrogate IMD 16 to receive the heart signals. Processor 80 may store heart signals within memory 82, and retrieve stored heart signals from memory 82. Processor 80 may also generate and store marker codes indicative of different cardiac events that sensing module 86 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

In some examples, IMD 16 may signal programmer 24 to further communicate with and pass the alert through a network such as the Medtronic CareLink® Network developed by Medtronic, Inc., of Minneapolis, Minn., or some other network linking patient 14 to a clinician. In this manner, a computing device or user interface of the network may be the external computing device that delivers the alert, e.g., patient metric data or heart failure risk score, to the user.

The various components of IMD 16 are coupled to power source 90, which may include a rechargeable or non-rechargeable battery. A non-rechargeable battery may be capable of holding a charge for several years, while a rechargeable battery may be inductively charged from an external device, e.g., on a daily or weekly basis. In other examples, power source 90 may include a supercapacitor.

In alternative embodiments, IMD 16 may automatically provide therapy to patient 14 based on the heart failure risk score and/or one of the patient metrics. For example, IMD 16 or another device may include a drug pump that delivers a dose of medication, e.g., nitroglycerin, to alleviate the imminent or present heart failure conditions. This drug pump may be in addition to or in place of electrical stimulation therapy devices. In other examples, IMD 16 may deliver pacing therapy to try and reduce the heart failure symptoms.

FIG. 4 is a functional block diagram illustrating an example configuration of external programmer 24. As shown in FIG. 4, programmer 24 may include a processor 100, memory 102, user interface 104, telemetry module 106, and power source 108. Programmer 24 may be a dedicated hardware device with dedicated software for programming of IMD 16. Alternatively, programmer 24 may be an off-the-shelf computing device running an application that enables programmer 24 to program IMD 16.

A user may use programmer 24 to select therapy programs (e.g., sets of stimulation parameters), generate new therapy programs, modify therapy programs through individual or global adjustments or transmit the new programs to a medical device, such as IMD 16 (FIG. 1). The clinician may interact with programmer 24 via user interface 104, which may include display to present graphical user interface to a user, and a keypad or another mechanism for receiving input from a user. In addition, the user may receive an alert or notification from IMD 16 indicating the heart failure risk score and/or patient metrics via programmer 24.

Processor 100 can take the form one or more microprocessors, DSPs, ASICs, FPGAs, programmable logic circuitry, or the like, and the functions attributed to processor 100 herein may be embodied as hardware, firmware, software or any combination thereof. Memory 102 may store instructions that cause processor 100 to provide the functionality ascribed to programmer 24 herein, and information used by processor 100 to provide the functionality ascribed to programmer 24 herein. Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient.

Programmer 24 may communicate wirelessly with IMD 16, such as using RF communication or proximal inductive interaction. This wireless communication is possible through the use of telemetry module 106, which may be coupled to an internal antenna or an external antenna. An external antenna that is coupled to programmer 24 may correspond to the programming head that may be placed over heart 12, as described above with reference to FIG. 1. Telemetry module 106 may be similar to telemetry module 88 of IMD 16 (FIG. 4).

Telemetry module 106 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16.

In this manner, telemetry module 106 may receive an alert or notification of the heart failure risk score from telemetry module 88 of IMD 16. The alert may be automatically transmitted, or pushed, by IMD 16 when the heart failure risk score becomes critical. In addition, the alert may be a notification to a healthcare professional, e.g., a clinician or nurse, of the risk score and/or an instruction to patient 14 to seek medical treatment for the potential heart failure condition. In response to receiving the alert, user interface 104 may present the alert to the healthcare professional regarding the risk score or present an instruction to patient 14 to seek medical treatment.

Either in response to pushed heart failure information, e.g., the risk score or patient metrics, or requested heart failure information, user interface 104 may present the patient metrics and/or the heart failure risk score to the user. In some examples, user interface 104 may also highlight each of the patient metrics that have exceeded the respective one of the plurality of metric specific thresholds. In this manner, the user may quickly review those patient metrics that have contributed to a critical heart failure risk score.

Upon receiving the alert via user interface 104, the user may also interact with user interface 104 to cancel the alert, forward the alert, retrieve data regarding the heart failure risk score (e.g., patient metric data), modify the metric specific thresholds used to determine the risk score, or conduct any other action related to the treatment of patient 14. In some examples, the clinician may be able to review raw data to diagnose any other problems with patient 14. User interface 104 may even suggest treatment along with the alert, e.g., certain drugs and doses, to minimize symptoms and tissue damage that could result from heart failure. User interface 104 may also allow the user to specify the type and timing of alerts based upon the severity or criticality of the heart failure risk score. In addition to the heart failure risk score, user interface 104 may also provide the underlying parameters to allow the clinician to monitor therapy efficacy and remaining patient conditions.

In some examples, processor 100 of programmer 24 and/or one or more processors of one or more networked computers may perform all or a portion of the techniques described herein with respect to processor 80 and IMD 16. For example, processor 100 or a metric detection module within programmer 24 may analyze patient metrics to detect those metrics exceeding thresholds and to generate the heart failure risk score.

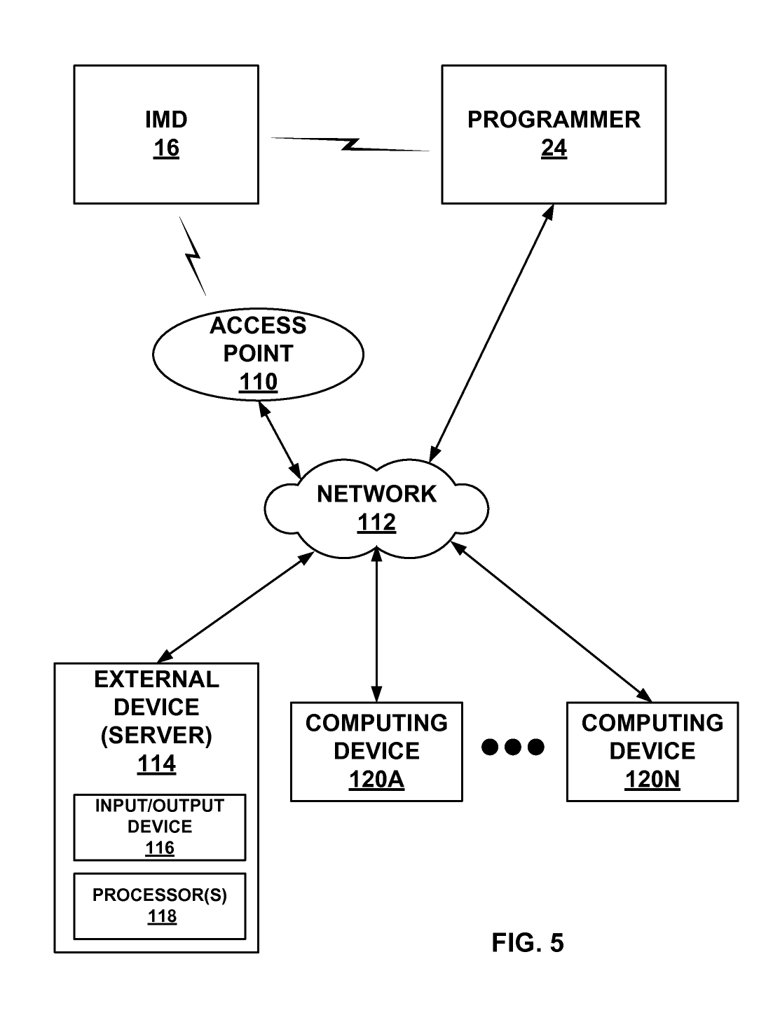

FIG. 5 is a block diagram illustrating an example system that includes an external device, such as a server 114, and one or more computing devices 120A-120N, that are coupled to the IMD 16 and programmer 24 shown in FIG. 1 via a network 112. Network 112 may be used to transmit an alert of the heart failure risk score from IMD 16 to another external computing device. In this example, IMD 16 may use its telemetry module 88 to communicate with programmer 24 via a first wireless connection, and to communication with an access point 110 via a second wireless connection. In the example of FIG. 5, access point 110, programmer 24, server 114, and computing devices 120A-120N are interconnected, and able to communicate with each other, through network 112. In some cases, one or more of access point 110, programmer 24, server 114, and computing devices 120A-120N may be coupled to network 112 through one or more wireless connections. IMD 16, programmer 24, server 114, and computing devices 120A-120N may each comprise one or more processors, such as one or more microprocessors, DSPs, ASICs, FPGAs, programmable logic circuitry, or the like, that may perform various functions and operations, such as those described herein.

Access point 110 may comprise a device that connects to network 112 via any of a variety of connections, such as telephone dial-up, digital subscriber line (DSL), or cable modem connections. In other examples, access point 110 may be coupled to network 112 through different forms of connections, including wired or wireless connections. In some examples, access point 110 may be co-located with patient 14 and may comprise one or more programming units and/or computing devices (e.g., one or more monitoring units) that may perform various functions and operations described herein. For example, access point 110 may include a home-monitoring unit that is co-located with patient 14 and that may monitor the activity of IMD 16. In some examples, server 114 or computing devices 120 may control or perform any of the various functions or operations described herein, e.g., generate a heart failure risk score based on the patient metric comparisons or create patient metrics from the raw metric data.

In some cases, server 114 may be configured to provide a secure storage site for archival of patient metric data and heart failure risk scores that has been collected and generated from IMD 16 and/or programmer 24. Network 112 may comprise a local area network, wide area network, or global network, such as the Internet. In some cases, programmer 24 or server 114 may assemble sensing integrity information in web pages or other documents for viewing by and trained professionals, such as clinicians, via viewing terminals associated with computing devices 120. The system of FIG. 5 may be implemented, in some aspects, with general network technology and functionality similar to that provided by the Medtronic CareLink® Network developed by Medtronic, Inc., of Minneapolis, Minn.

In the manner of FIG. 5, computing device 120A or programmer 24, for example, may be remote computing devices that receive and present heart failure risk scores from IMDs of multiple patients so that a clinician may prioritize the patients needing treatment immediately. In other words, the clinician may triage patients by analyzing the heart failure risk scores of multiple patients. The computing device may use its communication module to receive the heart failure risk scores from multiple IMDs via network 112. In this manner, each heart failure risk score is representative of one the patients. Although the IMDs may transmit the heart failure risk score at any time, generally the IMDs may transmit heart failure risk scores that are critical. A processor within the remote computing device may then automatically rank each of the patients based on each of the heart failure risk scores and the user interface may present the list of ranked patients to the clinician. Generally, the list will start with the most critical patient at the top. This method may useful for healthcare professionals making house calls, serving patients within a nursing home, or any other circumstance in which a professional treats many patients.

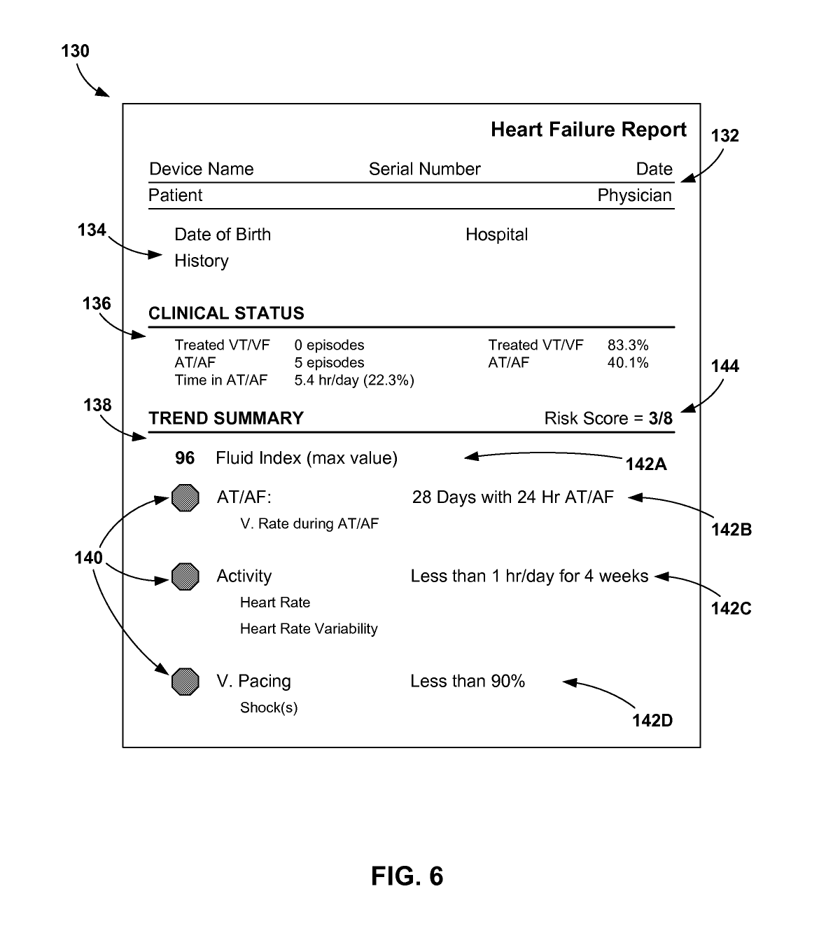

FIG. 6 illustrates an example screen 130 of user interface 104 that includes a trend summary 138 of patient metrics indicating a risk of heart failure. Although screen 130 is described as being presented on user interface 104 of programmer 24, screen 130 may be presented on any user interface of any device used by a healthcare professional. As shown in FIG. 6, screen 130 is a heart failure report that includes identification data 132 and patient history data 134. Identification data 132 includes items such as the patient name, the device name, the serial number of IMD 16, the date, and even the physician name. Patient history data 134 may be relevant data that may help in the treatment of patient 14.

Screen 130 also includes clinical status 136 that includes information regarding the stimulation therapy delivered by IMD 16. Screen 130 also presents trend summary 138. Trend summary 138 presents a quick snapshot of certain patient metrics that are exceeding their respective metric thresholds to contribute to the heart failure risk score shown as risk score 144. Critical indicator 140 is provided to remind the user that each of the patient metrics with critical indicator 140 is contributing to the heart failure risk score because the metric threshold has been met or exceeded.

In the example of FIG. 6, trend summary 138 presents four patient metrics 142A, 142B, 142C, and 142D (collectively “patient metrics 142”). Thoracic fluid index metric 142A indicates a maximum detected value of 96. Although thoracic fluid index metric 142A is not contributing to risk score 144 in this example, it is provided because it is an important indicator of thoracic fluid volume and potential heart failure. Atrial fibrillation duration 142B indicates that patient 14 has had 28 days of atrial fibrillation or atrial tachycardia for 24 hours. Activity metric 142C indicates that patient 14 has been active for less than 1 hour per day for the last 4 weeks. In addition, ventricular pacing metric 142D (e.g., a cardiac resynchronization therapy percentage) indicates that IMD 16 has been pacing heart 12 less than 90 percent of the time. As patient metrics 142 indicate, information may be given that is more specific than just a threshold has been exceeded. The actual observed patient metric data, or summary of the data, may be presented in trend summary 138.

Since each of patient metrics 142B-D has exceeded their respective metric specific threshold, critical indicator 140 is provided for each metric. The user then knows that heart failure risk score 144 is generated with these critical patient metrics. Heart failure risk score 144 may also indicate via highlighting or other indication when the risk score 144 is critical. Although heart failure risk score 144 is provided as a fraction of the critical patient metrics over the total number of observed metrics, risk score 144 may be provided as a percentage or even weighted average of the critical patient metrics.

Although screen 130 may be a passively presented informational screen, screen 130 may be interactive. The user may select areas of screen 130 to view more details about any of patient metrics 142, for example. Screen 130, in other examples, may provide scroll bars, menus, and navigation buttons to allow the user to view additional information, adjust therapy, adjust metric parameters, or perform other operations related to the treatment of patient 14 with the patient metrics and risk score.

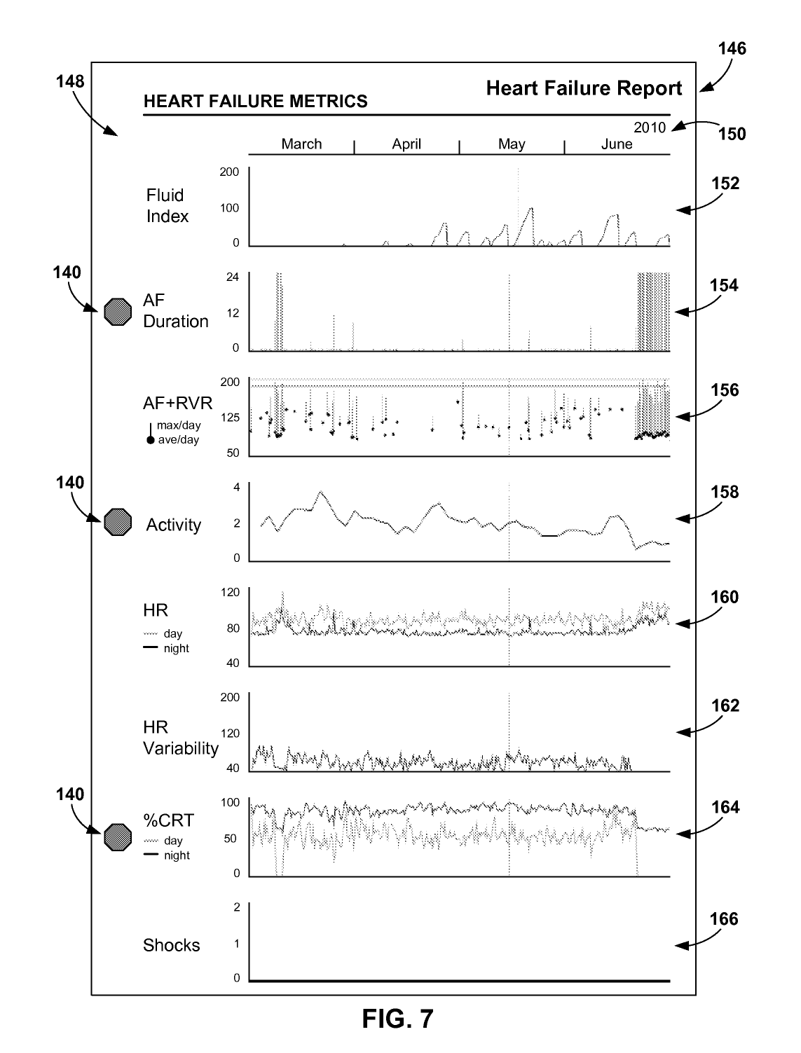

FIG. 7 illustrates an example screen 146 of user interface 104 that includes data from all of the patient metrics used to generate the heart failure risk score. Although screen 146 is described as being presented on user interface 104 of programmer 24, screen 130 may be presented on any user interface of any device used by a healthcare professional. As shown in FIG. 7, screen 146 provides another heart failure report, similar to screen 130 of FIG. 6. Screen 146 provides heart failure metrics 148 that include those patient metrics used to generate the heart failure risk score. Included are the metric data for eight patient metrics 152, 154, 156, 158, 160, 162, 164, and 166. Timeline 150 indicates for which months the data is representative in all the metric graphs. Although this four month period may be the evaluation window, timeline 150 may cover many evaluation windows. For example, the evaluation window may be equal to one month, such that the risk score is reviewed after the evaluation window expires. In addition, the user may move through time with an interactive timeline 150 in other examples. Although not presented in screen 146, the heart failure risk score may also be presented.

Thoracic fluid index metric 152 is labeled as “Fluid Index.” Thoracic fluid index metric 152 illustrates that the thoracic fluid index has been periodically raising and lowering over the months of May and June. In one example, the thoracic fluid index threshold may be approximately 60 ohms. However, the thoracic fluid index threshold may be generally between approximately 40 ohms and 200 ohms. Atrial fibrillation duration metric 154 is labeled “AF Duration” and indicates how many hours each day that the patient endured atrial fibrillation. As shown, atrial fibrillation duration metric 154 includes critical indicator 140 because of the days of atrial fibrillation shown at the end of June. An example atrial fibrillation duration threshold may be approximately 6 hours. However, the atrial fibrillation duration threshold may be set generally between approximately 1 hour and 24 hours. Ventricular contraction metric 156 is labeled “AF+RVR” and indicates the ventricular contraction rate during atrial fibrillation. The graph of ventricular contraction metric 156 provides the average ventricular contraction rate for each day and also the maximum ventricular contraction rate observed during each day. Generally, the ventricle contraction rate threshold may be set between approximately 70 beats per minute and 120 beats per minute for 24 hours. In one example, the ventricular contraction rate threshold may be approximately equal to 90 beats per minute for 24 hours. In other examples, the duration of 24 hours may be shorter or longer.

Activity metric 158 also is highlighted with critical indicator 140. Activity metric 158 is labeled “Activity” and indicates how many hours the patient is active each day. Lower activity levels may be a risk factor for heart failure, and the graph of activity metric 158 indicates that patient 14 has been less active at the end of June. In this manner, the patient metric of activity may be a metric where exceeding the metric specific threshold includes dropping below the threshold. In one example, the patient activity threshold may be approximately equal to 1 hour per day for seven consecutive days. In other examples, the threshold may be set to more or less time over a different duration. Instead of hours per day, other examples of activity metric 158 may provide durations of certain postures, e.g., lying down, sitting up, or standing. In general, activity metric 158 may include measurements of the rigor of patient activity and/or the amount of time patient 14 is active.

Screen 148 also provides for heart rate metrics. Heart rate metric 160 is labeled “HR” and indicates separate graphs for each of the nighttime heart rate and daytime heart rate. In some examples, the nighttime heart rate may be more indicative of heart failure risk. Generally, the nighttime hear rate threshold may be set to between approximately 70 beats per minute and 120 beats per minute for a certain period of time. In one example, the nighttime heart rate threshold may be approximately 85 beats per minute for seven consecutive days. Heart rate variability metric 162 is labeled “HR Variability” and indicates the degree of change in heart rate throughout the day. Since lower heart rate variability may indicate an increased sympathetic tone detrimental to blood flow through the vasculature, heart rate variability may also be a patient metric where exceeding the metric specific threshold includes dropping below the threshold. In one example, the heart rate variability threshold may be set to approximately 60 milliseconds for seven consecutive days, but other variability thresholds may also be used.

In addition, screen 148 may also provide a few patient metrics derived from therapy delivered to patient 14. Therapy percentage metric 164 is labeled “% CRT” and indicates the percentage of time each day and night that IMD 16 is delivering a cardiac resynchronization therapy, e.g., pacing therapy. Lower percentages of therapy may indicate diminished blood flow through the vasculature. Generally, the cardiac resynchronization therapy percentage threshold may be set to between 70 percent and 100 percent for a given period of time. In one example, the cardiac resynchronization therapy percentage threshold may be set to approximately 90 percent for five of seven consecutive days. Since the nighttime therapy percentage is less than 90 percent, critical indicator 140 is used to highlight therapy percentage metric 164. In other examples, a ventricular pacing percentage may be monitored for patients receiving pacing therapy with dual or single chamber pacing devices. Increased ventricular pacing may increase the risk of heart failure in some patients due to desynchronization of ventricular contractions in the heart. Further, shock metric 166 is labeled “Shocks” and indicates the number of electrical shock events, e.g., cardioversion or defibrillation, endured by patient 14. As shown in FIG. 7, patient 14 has not been subjected to any shock therapy. Although the threshold may be set to a different value, the electrical shock threshold may generally be set to approximately 1 electrical shock.

Since each of patient metrics 154, 158, and 164 have exceeded their respective metric specific threshold, critical indicator 140 is provided for each metric. In addition to, or in place of, critical indicators 140, patient metrics may be highlighted with a different text color, circles or boxes surround each metric, or some other indication of the critical level of each metric. In other examples, other patient metrics may be presented in heart failure metrics 148, e.g., blood pressure, lung volume, lung density, or respiration rate, weight, sleep apnea burden derived from respiration, temperature, ischemia burden, sensed cardiac event intervals, and troponin and/or brain natriuretic peptide (BNP) levels.

Although screen 148 may be a passively presented informational screen, screen 148 may be interactive. The user may select areas of screen 148 to view more details about any of the presented patient metrics, for example. The user may also move to different time periods with timeline 150. Screen 130, in other examples, may provide scroll bars, menus, and navigation buttons to allow the user to view additional information, adjust therapy, adjust metric parameters, or perform other operations related to the treatment of patient 14 with the patient metrics and risk score. Further, the user may interact with the graph of each patient metric to expand the graph and view more details of the graph, perhaps even individual values.

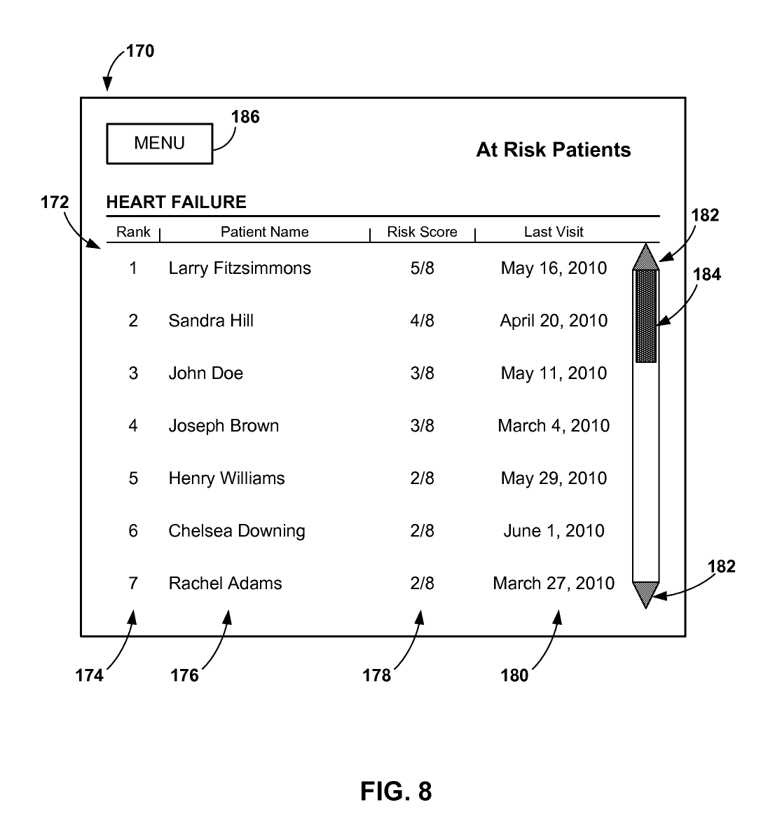

FIG. 8 illustrates an example user interface 170 that includes a list 172 of patients 176 ranked by severity of their heart failure risk score 178. User interface 170 may be a user interface of an external computing device, but user interface 170 may also be an example of user interface 104 of programmer 24 if programmer 24 is capable of receiving risk scores from multiple patients. As shown in the example of FIG. 8, user interface 170 presents list 172 of patients with critical heart failure risk scores. A communication module of the computing device has already received the risk scores from the IMDs of multiple patients and the processor has automatically ranked the patients by risk score.

User interface 170 includes list 172, scroll arrows 182, scroll bar 184, and menu 186. The user may select either of scroll arrows 182 to navigate through list 172 or select and move scroll bar 184 to navigate to other portions of list 172 not shown within the viewable field of the list. List 172 includes four data fields. Rank 174 indicates the severity rank of the patient, patients 176 includes the name of each patient in the list, risk scores 178 provides the received heart failure risk scores for each patient, and visit date 180 provides the date of the last visit between the patient and a healthcare professional.

As shown in the example of FIG. 8, the patient “Larry Fitzsimmons” has been ranked first because he has the highest, or most critical, risk score of all of the patients at “⅝”. List 172 thus allows a healthcare professional to triage patients and give attention to the patients most needing the treatment. However, list 172 may also be sortable by patient name or last visit if desired by the clinician. In other examples, list 172 may be presented one patient at a time. In other words, user interface 170 may force the user to view the most at risk patients first, one at a time.

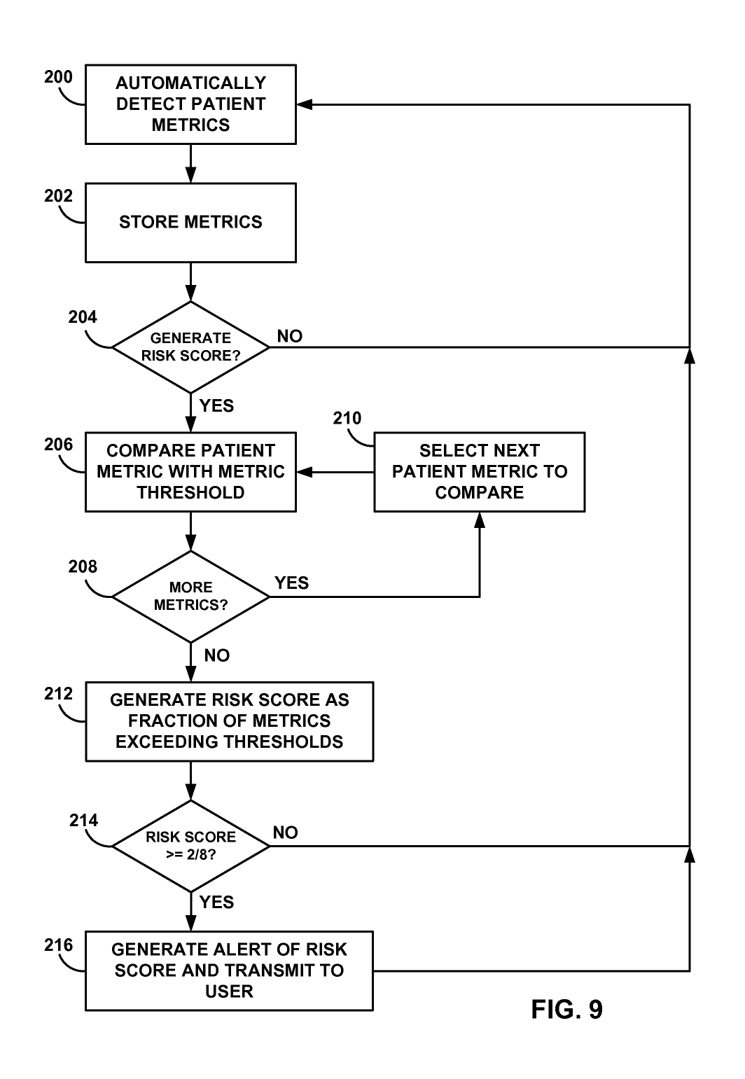

FIG. 9 is a flow diagram of an example method for generating heart failure risk scores from patient metrics. FIG. 9 will be described with IMD 16 both detecting patient metrics and generating heart failure risk scores for the patient, but other examples of the same technique may be applied to other devices (e.g. programmer 24 or an external computing device).

As shown in FIG. 9, metric detection module 92 automatically detects the patient metrics from various electrodes, sensors, and therapy information (200). Metric detection module 92 then stores the patient metrics in metric data 85 of memory 82 (202). If processor 80 does not need to generate the heart failure risk score (“NO” branch of block 204), metric detection module 92 continues to detect patient metrics (200). In some examples, processor 80 may only generate the risk score after an evaluation window expires. For example, if the evaluation window is one month, processor 80 may only generate the risk score after the month expires. However, processor 80 may generate and transmit the risk score as frequent as every hour or as infrequent as several months. If processor 80 is to generate the heart failure risk score (“Yes” branch of block 204), processor 80 compares one of the patient metrics with the metric specific threshold of that metric (206). If there are more patient metrics to compare (“Yes” branch of block 208), processor 80 selects the next patient metric to compare (210) and compares the metric to its threshold (206).

Once there are no more patient metrics to compare (“NO” branch of block 208), processor 80 generates the heart failure risk score as a fraction of metrics exceeding their thresholds as determined in the comparison step (212). In other examples, the risk score may be generated as a percentage or other value. If the risk score is less than the predetermined number of two patient metrics exceeding the eight total patient metrics (“NO” branch of block 214), then processor 80 continues with metric detection module 92 detecting the patient metrics. In other examples, a different predetermined number or total patient metrics maybe used.

If the risk score is equal to or greater than 2 of 8 (“YES” branch of block 214), processor 80 generates an alert of the risk score and transmits the alert to the user via telemetry module 88 (216). As described herein, the alert may be transmitted as soon as communication is possible to another device or access point. Alternatively, the heart failure risk score may only be transmitted when requested by a user. In some examples the alert may include detailed information regarding the patient metrics included in the risk score.

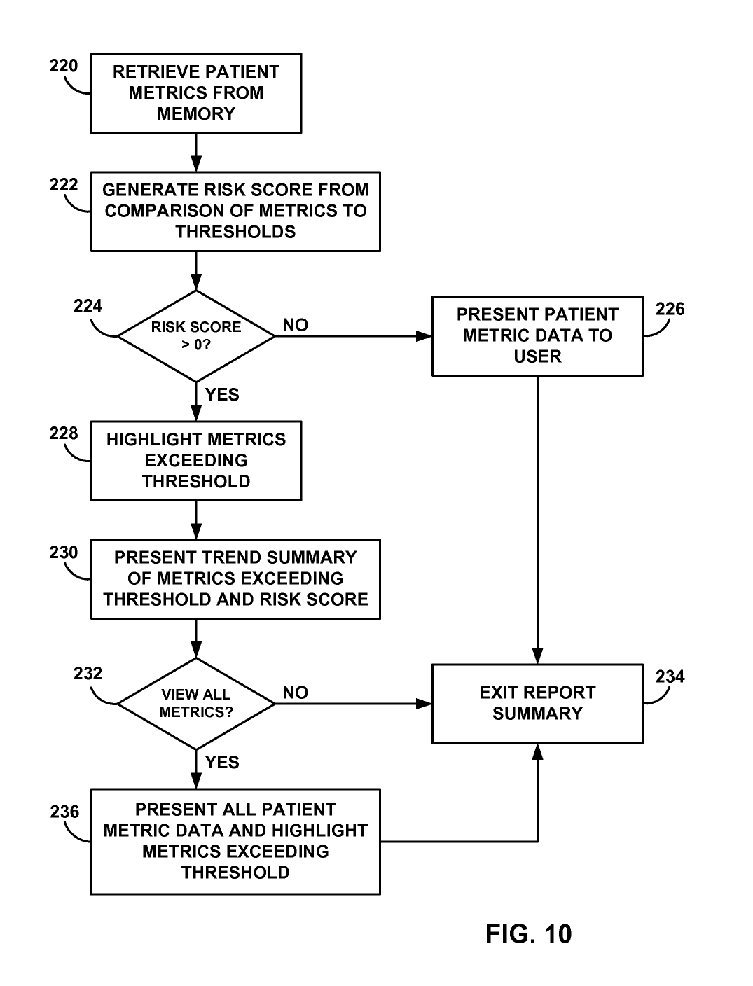

FIG. 10 is a flow diagram of an example method for presenting heart failure risk scores and patient metric data to a user. Although FIG. 10 is generally described with respect to IMD 16 and programmer 24, other computing devices may be used in other examples. As shown in FIG. 10, processor 100 retrieves patient metrics from memory 82 of IMD 16 (220). Processor 100 then generates the heart failure risk score by comparing each of the patient metrics to their respective metric specific thresholds (222). If the risk score is not greater than zero (“NO” branch of block 224), user interface 104 presents the patient metric data to the user without highlighting any metrics (226). For example, the patient metric data may be presented like the example of screen 146 of FIG. 7.

If the risk score is greater than zero (“YES” branch of block 224), user interface 104 highlights each of the patient metrics that exceed their respective threshold and contribute to the predetermined number of the heart failure risk score (228). User interface 104 then presents the trend summary of all of the patient metrics exceeding thresholds and the heart failure risk score (230). This presentation may be similar to screen 130 of FIG. 6. If the user does not want to view all of the patient metrics (“NO” branch of block 232), then user interface 104 exits the report summary and moves to the previous menu (234). If the user does want to view the other metrics (“YES” branch of block 236), then user interface 104 presents all of the patient metric data as graphs and still highlights those metrics exceeding their threshold, e.g., FIG. 7 (236). Once the user is done reviewing the metrics, user interface exits the summary report and moves to the previous menu (234).

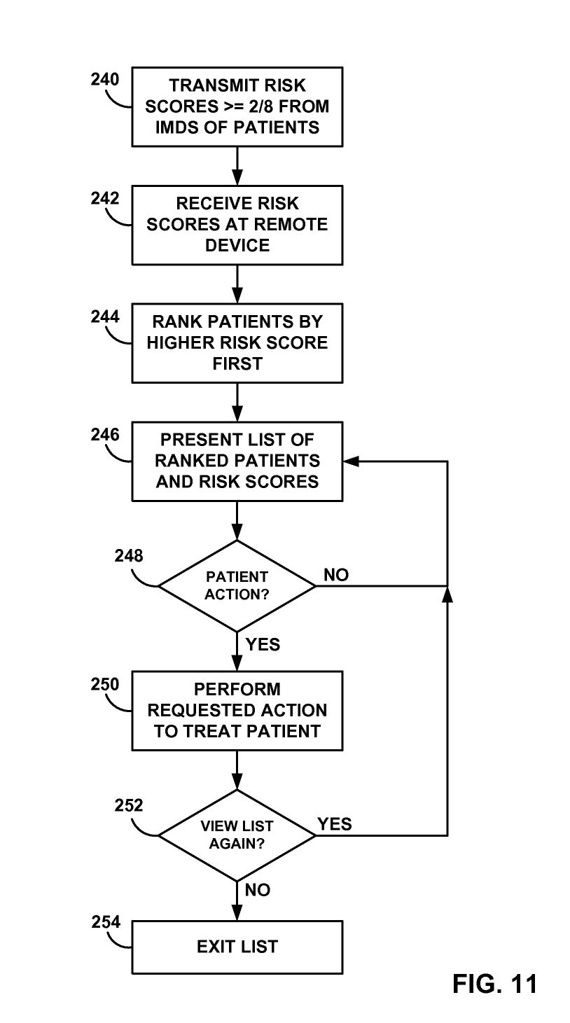

FIG. 11 is a flow diagram of an example method for presenting a user with a ranked list of patients based on the heart failure risk score of each patient. Although the technique of FIG. 11 is generally directed to an external computing device remote of patient 14, the technique may be implemented by any device capable of receiving heart failure risk scores from multiple patients. As shown in FIG. 11, the IMD for each patient in the care of the user transmits (or pushes) alerts with heart failure risk scores greater than or equal to 2 critical metrics out of 8 total metrics (240). However, the technique may use risk scores set to any predetermined number, even if they are patient specific. In other words, any critical risk when the predetermined number has been satisfied score may be transmitted.

The communication module of the external computing device receives each of the risk scores from each patient IMD (242). The computing device processor next analyzes the heart failure risk scores and ranks each patient by the highest, or most critical, risk score first (244). The computing device user interface then presents the list of the ranked patients and each respective risk score (246). As long as no patient action is requested by the user (“NO” branch of block 248), the user interface continues to present the list of ranked patients (246). If a patient action has been requested by the user (“YES” branch of block 248), then the computing device performs the requested action to treat the patient (250). This action may be scheduling a clinic visit, ordering medication, or even dispatching emergency personnel to treat the patient. If the user requests to view the list again (“YES” branch of block 252), the user interface again presents the list to the user (246). If the user does not wish to view the list again (“NO” branch of block 252), the user interface exists the list (254).

In other examples, the user may interact with the user interface to conduct further activities. For example, the external computing device may be capable of retrieving and presenting the patient metric data of each listed patient, calling the patient, programming therapy parameters of the remote IMD, adjusting metric instructions or metric specific thresholds, or even modifying the rules for generating the heart failure risk scores or transmitting the risk score alerts to the external computing device.

The techniques described herein allow an IMD to monitor several patient metrics that can be used to predict heart failure in a patient. A heart failure risk score may be automatically generated using the data stored from the patient metrics, and a risk score meeting the predetermined number of metrics exceeding a threshold may indicate a high likelihood of heart failure. The heart failure risk score and/or other metric data may be reviewed by a clinician, even remotely. In this manner, the clinician may be able to continually monitor the patient's status with automatically detected patient metrics and automatically generated risk score. At bottom, a patient may receive prompt medical treatment to avoid severe conditions related to heart failure, including death. In other examples, the heart failure risk score may allow the clinician to view a list of patients needing treatment ranked according to their heart failure risk score. These techniques may aid in earlier treatment, minimized patient complications and hospital stays, and a higher quality of life for those patients at risk of heart failure

Various examples have been described that include detecting and storing patient metrics and generating heart failure risk scores. These examples include techniques for identifying higher risk patients with the risk score. In addition, an alert of risk scores may be remotely delivered to a healthcare professional for earlier diagnosis and treatment of hearth failure. Any combination of detection and notification of heart failure risk is contemplated. These and other examples are within the scope of the following claims.

1. A method comprising:
storing a plurality of automatically detected patient metrics within an implantable medical device;
comparing each of the plurality of automatically detected patient metrics to one of a plurality of metric specific thresholds; and
automatically generating a heart failure risk score based on the comparison, wherein the heart failure risk score indicates an increased risk of heart failure when a predetermined number of the plurality of automatically detected patient metrics each exceed the respective one of the plurality of metric specific thresholds.

2. The method of claim 1, wherein:
the predetermined number of the plurality of automatically detected patient metrics is two automatically detected patient metrics each exceeding the respective one of the plurality of metric specific thresholds; and
the plurality of automatically detected patient metrics includes at least 8 different automatically detected patient metrics.

3. The method of claim 1, wherein:
the plurality of automatically detected patient metrics comprises at least two of a thoracic fluid index, an atrial fibrillation duration, a ventricular contraction rate during atrial fibrillation, a patient activity, a nighttime heart rate, a heart rate variability, a cardiac resynchronization therapy percentage, a ventricular pacing percentage, and an electrical shock event; and

4. The method of claim 1, wherein the plurality of metric specific thresholds comprise at least two of a thoracic fluid index threshold of approximately 60 ohms, an atrial fibrillation duration threshold of approximately 6 hours, a ventricular contraction rate threshold approximately equal to 90 beats per minute for 24 hours, a patient activity threshold approximately equal to 1 hour per day for seven consecutive days, a nighttime heart rate threshold of approximately 85 beats per minute for seven consecutive days, a heart rate variability threshold of approximately 60 milliseconds for seven consecutive days, a cardiac resynchronization therapy percentage threshold of 90 percent for five of seven consecutive days, and an electrical shock threshold of 1 electrical shock.

5. The method of claim 1, further comprising automatically detecting each of the plurality of automatically detected patient metrics with the implantable medical device, wherein the automatic detection comprises at least one of measuring a thoracic impedance, analyzing an electrogram of a heart, monitoring an electrical stimulation therapy delivery, and sensing a patient activity.

6. The method of claim 1, further comprising:
automatically generating an alert in response to the automatically generated heart failure risk score, wherein the alert comprises the heart failure risk score; and
transmitting the alert to a user.

7. The method of claim 1, further comprising:
presenting the plurality of automatically detected patient metrics and the heart failure risk score to a user; and
highlighting each of the plurality of automatically detected patient metrics that exceed the respective one of the plurality of metric specific thresholds.

8. The method of claim 1, further comprising:
receiving a plurality of heart failure risk scores at a remote computing device, wherein each of the plurality of heart failure risk scores is representative of one of a plurality of patients;
automatically ranking the plurality of patients based on each of the plurality of heart failure risk scores; and
presenting a list of the ranked plurality of patients to a healthcare professional.

9. The method of claim 1, wherein the implantable medical device is configured to deliver electrical stimulation therapy to a heart of a patient.

10. A system comprising:
a memory of an implantable medical device that stores a plurality of automatically detected patient metrics; and
a metric detection module configured to compare each of the plurality of automatically detected patient metrics to one of a plurality of metric specific thresholds and automatically generate a heart failure risk score based on the comparison, wherein the heart failure risk score indicates an increased risk of heart failure when a predetermined number of the plurality of automatically detected patient metrics each exceed the respective one of the plurality of metric specific thresholds.

11. The system of claim 10, wherein:
the predetermined number of the plurality of automatically detected patient metrics is two automatically detected patient metrics each exceeding the respective one of the plurality of metric specific thresholds; and
the plurality of automatically detected patient metrics includes at least 8 different automatically detected patient metrics.

12. The system of claim 10, wherein the plurality of automatically detected patient metrics comprises at least two of a thoracic fluid index, an atrial fibrillation duration, a ventricular contraction rate during atrial fibrillation, a patient activity, a nighttime heart rate, a heart rate variability, a cardiac resynchronization therapy percentage, a ventricular pacing percentage, and an electrical shock event.

13. The system of claim 10, wherein the plurality of metric specific thresholds comprise at least two of a thoracic fluid index threshold of approximately 60 ohms, an atrial fibrillation duration threshold of approximately 6 hours, a ventricular contraction rate threshold approximately equal to 90 beats per minute for 24 hours, a patient activity threshold approximately equal to 1 hour per day for seven consecutive days, a nighttime heart rate threshold of approximately 85 beats per minute for seven consecutive days, a heart rate variability threshold of approximately 60 milliseconds for seven consecutive days, a cardiac resynchronization therapy percentage threshold of 90 percent for five of seven consecutive days, and an electrical shock threshold of 1 electrical shock.

14. The system of claim 10, wherein the metric detection module within the implantable medical device configured to at least one of measure a thoracic impedance, analyze an electrogram of a heart, monitor an electrical stimulation therapy delivery, and sense a patient activity.

15. The system of claim 10, further comprising a telemetry module, wherein:
the metric detection module automatically generates an alert in response to the automatically generated heart failure risk score, wherein the alert comprises the heart failure risk score; and
the telemetry module transmits the alert to a user.

16. The system of claim 10, further comprising a user interface configured to present the plurality of automatically detected patient metrics and the heart failure risk score to a user and highlight each of the plurality of automatically detected patient metrics that exceed the respective one of the plurality of metric specific thresholds.

17. The system of claim 10, further comprising a remote computing device that comprises:
a communication module that receives a plurality of heart failure risk scores, wherein each of the plurality of heart failure risk scores is representative of one of a plurality of patients;
a remote computing device processor that automatically ranks the plurality of patients based on each of the plurality of heart failure risk scores; and
a user interface that presents a list of the ranked plurality of patients to a healthcare professional.

18. The system of claim 10, wherein the implantable medical device comprises a signal generator configured to deliver electrical stimulation therapy to a heart of a patient.

19. A system comprising:
means for storing a plurality of automatically detected patient metrics within an implantable medical device;
means for comparing each of the plurality of automatically detected patient metrics to one of a plurality of metric specific thresholds; and
means for automatically generating a heart failure risk score based on the comparison, wherein the heart failure risk score indicates an increased risk of heart failure when a predetermined number of the plurality of automatically detected patient metrics each exceed the respective one of the plurality of metric specific thresholds.

20. The system of claim 20, further comprising:
means for automatically generating an alert in response to the automatically generated heart failure risk score, wherein the alert comprises the heart failure risk score; and
means for transmitting the alert to a user.
